SEQUENCE LISTING (67 sequences)

SEQ ID NO: 1 - DNA, 35 bp, synthetic construct
  source 1..35: mol_type = other DNA
ccctagaaagatagtctgcgtaaaattgacgcatg

SEQ ID NO: 2 - DNA, 63 bp, synthetic construct
  source 1..63: mol_type = other DNA
ccctagaaagataatcatattgtgacgtacgttaaagataatcatgcgtaaaattgacgcatg

SEQ ID NO: 3 - DNA, 44 bp, synthetic construct
  source 1..44: mol_type = other DNA
ccctagaaagataatcatattgtgacgtacgttaaagataatca

SEQ ID NO: 4 - DNA, 39 bp, synthetic construct
  source 1..39: mol_type = other DNA
ccctagaaagataatcatattgtgacgtacgttaaagat

SEQ ID NO: 5 - DNA, 34 bp, synthetic construct
  source 1..34: mol_type = other DNA
ccctagaaagataatcatattgtgacgtacgtta

SEQ ID NO: 6 - DNA, 29 bp, synthetic construct
  source 1..29: mol_type = other DNA
ccctagaaagataatcatattgtgacgta

SEQ ID NO: 7 - DNA, 24 bp, synthetic construct
  source 1..24: mol_type = other DNA
ccctagaaagataatcatattgtg

SEQ ID NO: 8 - DNA, 19 bp, synthetic construct
  source 1..19: mol_type = other DNA
ccctagaaagataatcata

SEQ ID NO: 9 - DNA, 17 bp, synthetic construct
  source 1..17: mol_type = other DNA
ccctagaaagataatca

SEQ ID NO: 10 - DNA, 515 bp, synthetic construct
  source 1..515: mol_type = other DNA
tccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctcgtccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccca

SEQ ID NO: 11 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 12 - DNA, 272 bp, synthetic construct
  source 1..272: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaa

SEQ ID NO: 13 - DNA, 248 bp, synthetic construct
  source 1..248: mol_type = other DNA
ttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 14 - DNA, 189 bp, synthetic construct
  source 1..189: mol_type = other DNA
catgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaa

SEQ ID NO: 15 - DNA, 274 bp, synthetic construct
  source 1..274: mol_type = other DNA
cattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatc

SEQ ID NO: 16 - DNA, 175 bp, synthetic construct
  source 1..175: mol_type = other DNA
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaaca

SEQ ID NO: 17 - DNA, 8920 bp, synthetic construct
  source 1..8920: mol_type = other DNA
ggcctaactggccggtacctgagtcttccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctcgtccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaagcttatcgataccgtcgacctcgagggggggcccggtacccaattcgccctatagtgagtcgtattaagatctaattaaccctcactaaagggaacaaaagctggagctccaccgcggtggcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagaactagtggatcccccgggctgcaggaattcgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 18 - DNA, 6474 bp, synthetic construct
  source 1..6474: mol_type = other DNA
taaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaagcttatcgataccgtcgacctcgagggggggcccggtacccaattcgccctatagtgagtcgtattaagatctaattaaccctcactaaagggaacaaaagctggagctccaccgcggtggcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttggggggggggctgtccctctagaactagtggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgc

SEQ ID NO: 19 - DNA, 5941 bp, synthetic construct
  source 1..5941: mol_type = other DNA
gaccactaccagcagaaacatgcgtcaattttacgcatgattatctttaacgtacgtcacaatatgattatctttctagggttaatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcatgagctgaagggcatcgagcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttggggggggggctgtccctctagaactagtggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgc

SEQ ID NO: 20 - DNA, 5897 bp, synthetic construct
  source 1..5897: mol_type = other DNA
gaccactaccagcagaaatatgattatctttctagggttaatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcatgagctgaagggcatcgagcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttggggggggggctgtccctctagaactagtggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgc

SEQ ID NO: 21 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASHDGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIASHDGGKQALETVQRLLPVLCQDHGLTPEQVVAIANNNGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 22 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIANNNGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNIGGKQALETVQRLLPVLCQDHGLTPEQVVAIASNGGGKQALETVQRLLPVLCQAHGLTPDQVVAIANNNGGKQALETVQRLLPVLCQAHGLTPAQVVAIASNIGGKQALETVQRLLPVLCQDHGLTPDQVVAIASNIGGKQALETVQRLLPVLCQDHGLTPEQVVAIASHDGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 23 - DNA, 11 bp, synthetic construct
  source 1..11: mol_type = other DNA
tgcagatgaac

SEQ ID NO: 24 - DNA, 85 bp, synthetic construct
  source 1..85: mol_type = other DNA
ccctagaaagataatcatatttgcagatgaacgcaatctcacggcgacgtacgttaaagataatcatgcgtaaaattgacgcatg

SEQ ID NO: 25 - DNA, 90 bp, synthetic construct
  source 1..90: mol_type = other DNA
ccctagaaagataatcatatttctgctgcagatgaacgcaatctcacggcgacgtacgttaaagataatcatgcgtaaaattgacgcatg

SEQ ID NO: 26 - DNA, 95 bp, synthetic construct
  source 1..95: mol_type = other DNA
ccctagaaagataatcatatttctgctggtatgcagatgaacgcaatctcacggcgacgtacgttaaagataatcatgcgtaaaattgacgcatg

SEQ ID NO: 27 - DNA, 100 bp, synthetic construct
  source 1..100: mol_type = other DNA
ccctagaaagataatcatatttctgctggtagtggttgcagatgaacgcaatctcacggcgacgtacgttaaagataatcatgcgtaaaattgacgcatg

SEQ ID NO: 28 - DNA, 105 bp, synthetic construct
  source 1..105: mol_type = other DNA
ccctagaaagataatcatatttctgctggtagtggtcggcgtgcagatgaacgcaatctcacggcgacgtacgttaaagataatcatgcgtaaaattgacgcatg

SEQ ID NO: 29 - DNA, 110 bp, synthetic construct
  source 1..110: mol_type = other DNA
ccctagaaagataatcatatttctgctggtagtggtcggcgagctgtgcagatgaacgcaatctcacggcgacgtacgttaaagataatcatgcgtaaaattgacgcatg

SEQ ID NO: 30 - DNA, 71 bp, synthetic construct
  source 1..71: mol_type = other DNA
ccctagaaagatagtctgcgtaaaattgacgcatgcatcgttacgcgtgccagttaaagataatcaatatt

SEQ ID NO: 31 - DNA, 76 bp, synthetic construct
  source 1..76: mol_type = other DNA
ccctagaaagatagtctgcgtaaaattgacgcatgcatctaagcgttacgcgtgccagttaaagataatcaatatt

SEQ ID NO: 32 - DNA, 81 bp, synthetic construct
  source 1..81: mol_type = other DNA
ccctagaaagatagtctgcgtaaaattgacgcatgcatgaaccctaagcgttacgcgtgccagttaaagataatcaatatt

SEQ ID NO: 33 - DNA, 86 bp, synthetic construct
  source 1..86: mol_type = other DNA
ccctagaaagatagtctgcgtaaaattgacgcatgcatcagatgaaccctaagcgttacgcgtgccagttaaagataatcaatatt

SEQ ID NO: 34 - DNA, 91 bp, synthetic construct
  source 1..91: mol_type = other DNA
ccctagaaagatagtctgcgtaaaattgacgcatgcatagctgcagatgaaccctaagcgttacgcgtgccagttaaagataatcaatatt

SEQ ID NO: 35 - DNA, 96 bp, synthetic construct
  source 1..96: mol_type = other DNA
ccctagaaagatagtctgcgtaaaattgacgcatgcatgtgcgagctgcagatgaaccctaagcgttacgcgtgccagttaaagataatcaatatt

SEQ ID NO: 36 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIASHDGGKQALETVQRLLPVLCQAHGLTPDQVVAIASNIGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASNGGGKQALETVQRLLPVLCQAHGLTPAQVVAIANNNGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 37 - DNA, 52 bp, unidentified
  source 1..52: mol_type = other DNA
tcctagtctctgataaaacagactttaaaccaacaaagatcaaaagagacaa

SEQ ID NO: 38 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNIGGKQALETVQRLLPVLCQDHGLTPDQVVAIANNNGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQALETVQRLLPVLCQDHGLTPDQVVAIASHDGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 39 - protein, 1071 aa, synthetic construct
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASNGGGKQALETVQRLLPVLCQDHGLTPEQVVAIANNNGGKQALETVQRLLPVLCQAHGLTPDQVVAIASNGGGKQALETVQRLLPVLCQAHGLTPAQVVAIASHDGGKQALETVQRLLPVLCQDHGLTPDQVVAIASNGGGKQALETVQRLLPVLCQDHGLTPEQVVAIASHDGGKQALETVQRLLPVLCQAHGLTPDQVVAIASNGGGKQALETVQRLLPVLCQAHGLTPAQVVAIASNGGGKQALETVQRLLPVLCQDHGLTPDQVVAIASNGGGKQALETVQRLLPVLCQDHGLTPEQVVAIASNGGGRPALESIVAQLSRPDPALAALTNDHLVALACLGGRPALDAVKKGLPHAPALIKRTNRRIPERTSHRVAGGGGSNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVASNAREIPEVLKNSRSRPVGTSMFCFDGPLTLVSYKPKPAKMVYLLSSCDEDASINESTGKPQMVMYYNQTKGGVDTLNQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 40 - DNA, 610 bp, synthetic construct
  source 1..610: mol_type = other DNA
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaattcctgagatatcgtcacctagctggcagagtggtatcgacgataagcttgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 41 - DNA, 392 bp, synthetic construct
  source 1..392: mol_type = other DNA
ttaaccctagaaagatagtctgcgtaaaattgacgcatgcatgtgcgagctcctagtctctcctaagcgttacgcgtgccagttaaagataatcaatattacaactacaacagccacaacgtctatatcatggccgacaagcagaagaacggcatcaaggtgaacttcaagatccgccacaacatcgaggacggcagcgtgaacggcgactgagatgtcctaaatgcacagcgacggattcgcgctatttagaaagagagagcaatatttcaagaatgcatgcgtcaattttacgcatgattatctttaacgtacgtcgccgtgagattgcaaaagagacaacagctcgccgaccactaccagcagaaatatgattatctttctagggttaa

SEQ ID NO: 42 - DNA, 365 bp, synthetic construct
  source 1..365: mol_type = other DNA
ttaaccctagaaagatagtctgcgtaaaattgacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaacatgcgtcaattttacgcatgattatctttaacgtacgtcacaatatgattatctttctagggttaa

SEQ ID NO: 43 - DNA, 321 bp, synthetic construct
  source 1..321: mol_type = other DNA
ttaaccctagaaagatagtctgcgtaaaattgacgcatgcattcttgaaatattgctctctctttctaaatagcgcgaatccgtcgctgtgcatttaggacatctcagtcgcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaaatatgattatctttctagggttaa

SEQ ID NO: 44 - DNA, 4401 bp, synthetic construct
  source 1..4401: mol_type = other DNA
ctagcggtctctttaaccctagaaagatagtctgcgtaaaattgacgcatgcattcttgaaatattgctctctctttctaaatgacgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcggcctccaaggcctgcaggagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtttagtcctgcaggtcggccgccacgaccggtgccaccatgactgaatacaaaccaactgttcgcctggcaactcgtgatgatgttccacgtgcagttcgcaccctggctgctgcatttgctgactaccctgcaacccgtcacactgtggacccagaccgccacattgaacgtgtgactgaactgcaggagctgttcctgacccgtgtgggcctggacattggcaaagtgtgggtggcagatgatggtgctgctgtggcagtgtggaccacccctgaatctgttgaagctggtgcagtgtttgctgagattggcccacgcatggcagaactgtctggcagccgcctggcagcacaacagcagatggaaggtctgctggcaccacaccgcccaaaagaacctgcttggttcctggcaactgtgggtgtgagccctgaccaccagggtaagggcctgggctctgcagtggtgctgcctggtgtggaagcagctgaacgtgcaggtgtgcctgctttcctggagacctcagctccacgcaacctgcctttctatgaacgcctgggcttcactgtgactgctgatgtggaagtgccagaaggcccacgcacttggtgcatgactcgcaaaccaggtgctagccttggctccggcgagggcaggggaagtcttctaacatgcggggacgtggaggaaaatcccggcccactcggctacccctacgacgtgcccgactacgccagcggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaactagatgctgtcgaccgatgcccttgagagccttcaacccagtcagctccttccggtgggcgcggggcaatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgactatcgtcgccgcacttattaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaagagacctgtacatacctaatgatttttatcaaaatcattaagttaaggtagatacacatcttgtcatatgatcaaatggtttcgccaaaaatcaataatcagacaacaagatgtgcgaactcgatattttacacgactctctttaccaattctaccacaacgtggatcagcagttcaacctgttgatagtacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccaaaaaaaagcccgctcattaggcgggctgcgcccttaagg

SEQ ID NO: 45 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRFDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 46 - DNA, 16 bp, synthetic construct
  source 1..16: mol_type = other DNA
ccctagaaagataatc

SEQ ID NO: 47 - DNA, 18 bp, synthetic construct
  source 1..18: mol_type = other DNA
ccctagaaagataatcat

SEQ ID NO: 48 - DNA, 20 bp, synthetic construct
  source 1..20: mol_type = other DNA
ccctagaaagataatcatat

SEQ ID NO: 49 - DNA, 21 bp, synthetic construct
  source 1..21: mol_type = other DNA
ccctagaaagataatcatatt

SEQ ID NO: 50 - DNA, 22 bp, synthetic construct
  source 1..22: mol_type = other DNA
ccctagaaagataatcatattg

SEQ ID NO: 51 - DNA, 23 bp, synthetic construct
  source 1..23: mol_type = other DNA
ccctagaaagataatcatattgt

SEQ ID NO: 52 - DNA, 25 bp, synthetic construct
  source 1..25: mol_type = other DNA
ccctagaaagataatcatattgtga

SEQ ID NO: 53 - DNA, 26 bp, synthetic construct
  source 1..26: mol_type = other DNA
ccctagaaagataatcatattgtgac

SEQ ID NO: 54 - DNA, 27 bp, synthetic construct
  source 1..27: mol_type = other DNA
ccctagaaagataatcatattgtgacg

SEQ ID NO: 55 - DNA, 28 bp, synthetic construct
  source 1..28: mol_type = other DNA
ccctagaaagataatcatattgtgacgt

SEQ ID NO: 56 - DNA, 30 bp, synthetic construct
  source 1..30: mol_type = other DNA
ccctagaaagataatcatattgtgacgtac

SEQ ID NO: 57 - DNA, 31 bp, synthetic construct
  source 1..31: mol_type = other DNA
ccctagaaagataatcatattgtgacgtacg

SEQ ID NO: 58 - DNA, 32 bp, synthetic construct
  source 1..32: mol_type = other DNA
ccctagaaagataatcatattgtgacgtacgt

SEQ ID NO: 59 - DNA, 33 bp, synthetic construct
  source 1..33: mol_type = other DNA
ccctagaaagataatcatattgtgacgtacgtt

SEQ ID NO: 60 - DNA, 35 bp, synthetic construct
  source 1..35: mol_type = other DNA
ccctagaaagataatcatattgtgacgtacgttaa

SEQ ID NO: 61 - DNA, 36 bp, synthetic construct
  source 1..36: mol_type = other DNA
ccctagaaagataatcatattgtgacgtacgttaaa

SEQ ID NO: 62 - DNA, 37 bp, synthetic construct
  source 1..37: mol_type = other DNA
ccctagaaagataatcatattgtgacgtacgttaaag

SEQ ID NO: 63 - DNA, 38 bp, synthetic construct
  source 1..38: mol_type = other DNA
ccctagaaagataatcatattgtgacgtacgttaaaga

SEQ ID NO: 64 - DNA, 40 bp, synthetic construct
  source 1..40: mol_type = other DNA
ccctagaaagataatcatattgtgacgtacgttaaagata

SEQ ID NO: 65 - DNA, 41 bp, synthetic construct
  source 1..41: mol_type = other DNA
ccctagaaagataatcatattgtgacgtacgttaaagataa

SEQ ID NO: 66 - DNA, 42 bp, synthetic construct
  source 1..42: mol_type = other DNA
ccctagaaagataatcatattgtgacgtacgttaaagataat

SEQ ID NO: 67 - DNA, 43 bp, synthetic construct
  source 1..43: mol_type = other DNA
ccctagaaagataatcatattgtgacgtacgttaaagataatc

(19) United States
(12) Patent Application Publication
(10) Pub. No.: US 2026/0021207 A1
(43) Pub. Date: Jan. 22, 2026

(54) SUPER MINIMAL INVERTED TERMINAL REPEAT (ITR) SEQUENCES AND USES THEREOF

(71) Applicant: Poseida Therapeutics, Inc., San Diego, CA (US)

(72) Inventors: Joseph S. LUCAS, San Diego, CA (US); Blair B. MADISON, San Diego, CA (US); Olga BATALOV, San Diego, CA (US)

(21) Appl. No.: 19/349,690

(22) Filed: Oct. 3, 2025

Related U.S. Application Data
(63) Continuation of application No. PCT/US2024/022983, filed on Apr. 4, 2024.
(60) Provisional application No. 63/494,300, filed on Apr. 5, 2023.

(51) Int. Cl. A61K 48/00; C12N 15/90

(52) U.S. Cl. CPC A61K 48/0058; A61K 48/0091; C12N 15/907; C12N 2800/90

(57) ABSTRACT

This disclosure generally relates to super minimal transposon inverted repeat sequence (ITR) polynucleotides, compositions comprising the polynucleotides and methods of using compositions comprising the polynucleotides for the ex vivo and in vivo delivery of nucleic acids to cells, in particular, in vivo delivery of therapeutic genes to treat genetic disorders or diseases.

### CROSS-REFERENCE TO RELATED APPLICATIONS

The present application is a continuation of International Patent Application No. PCT/US2024/022983, filed Apr. 4, 2024, which claims the benefit of U.S. Provisional Patent Application No. 63/494,300 filed Apr. 5, 2023, each of which is incorporated herein by reference in their entireties.

### REFERENCE TO SEQUENCE LISTING SUBMITTED ELECTRONICALLY

The instant application contains a Sequence Listing, which has been submitted electronically in XML file format, and is herein incorporated by reference into the specification in its entirety. The XML file containing the Sequence Listing XML is named “000218-0141-101-SL.xml,” was created on Oct. 3, 2025 and is 101,997 bytes in size.

### FIELD

This disclosure generally relates to super minimal transposon inverted repeat sequence (ITR) polynucleotides, compositions comprising the polynucleotides and methods of using compositions comprising the polynucleotides for the ex vivo and in vivo delivery of nucleic acids to cells, in particular, in vivo delivery of therapeutic genes to treat genetic disorders or diseases.

### BACKGROUND

Transposases may be used to introduce non-endogenous DNA sequences into genomic DNA, and are in many ways advantageous to other methods gene editing. However, there remains an unmet need for improvements to transposon ITR design to enhance ex vivo or in vivo transposase integration and/or excision activity.

The piggyBac (PB) transposase contains two distinct DNA binding domains that interact with the Inverted Terminal Repeat (ITR) sequences of a transposon. The DNA binding and dimerization domain (DDBD) binds sequence proximal to TTAA sites flanking the transposon, while the Cysteine Rich Domain (CRD) at the C-terminus of the protein binds sequence distal to the TTAA sites. Upon dimerization of the transposase, the DDBDs bind symmetrically to the ITRs, with the DDBD of one monomer binding the left end (LE) ITR and the DDBD of the second monomer binding the (RE) ITR, whereas the two CRDs of the dimer bind asymmetrically, with both binding to the LE ITR. The RE ITR is predicted, based on sequence homology, to comprise two CRD binding sites, suggesting a second dimer of PB transposase may bind distal to the first dimer. It is hypothesized that the proximal dimer catalyzes the transposition reaction, whereas the distal dimer stabilizes formation of the hairpin structure that brings the LE and RE together, a prerequisite for transposition to occur.

The DDBD and the CRD of the PB transposase bind to the ITRs in a sequence specific manner. The LE ITR comprises approximately 35 bp of DNA while the RE ITR is approximately 63 bp in length. The DDBD interacts with about 10 bp of DNA located 6 bp away from the TTAA sequences flanking the transposon. The binding of the DDBD is symmetrical with the DDBD of one transposase monomer binding the LE ITR and the DDBD of the second monomer binding the RE ITR. The CRD domains of the dimer bind to a 19 bp sequence of the LE ITR found immediately distal to the DDBD binding site. CRD binding is asymmetric, with both CRD domains of the dimer interacting with the 19 bp sequence on the LE ITR only. The RE ITR comprises a second DDBD binding sequence followed by a 19 bp CRD binding sequence starting 34 bp in from the TTAA, a potential second dimer PB transposase binding site.

In nature, the 5′ untranslated region (UTR) and 3′UTR are found directly adjacent to the LE and RE ITRs, respectively, of the transposon, with the coding sequence of the PB transposase juxtaposed between the UTRs. The PB 5′UTR and 3′UTR are not required for transposition; however, the 5′UTR and 3′UTR, or at least a portion of them, are often retained in recombinant transposon constructs for delivering DNA as it is believed that they increase the rate of transposition through an undescribed mechanism. Based on sequence homology, the 5′UTR is hypothesized to contain a sequence that shares a reasonable degree of similarity to a PB transposase binding site.

Generally, the LE ITR and 5′UTR together comprise 328 bp of DNA, whereas the RE ITR and 3′UTR together comprise 359 bp. In some transposon constructs, the end of the 5′UTR distal to the TTAA may be truncated, resulting in a LE ITR+5′UTR length of 309 bp. Similarly, the end of the 3′UTR distal to the TTAA has been truncated resulting in a RE ITR+3′UTR length of 238 bp. The combined 309 bp LE ITR/5′UTR and 238 bp RE ITR/3′UTR are often referred to as “Full ITRs.”

Transposon ITR sequences in which the 5′UTR has been deleted (generating a 35 bp LE ITR) and ITR sequences in which the 3′UTR has been deleted (generating a 63 bp RE ITR) are referred to as “Minimal LE PB ITRs” and “Minimal RE ITRs,” respectively. Such minimal ITRs are provided herein.

### SUMMARY

In one aspect, provided herein is a polynucleotide encoding a transposon, comprising a super minimal piggyBac right end (RE) inverted terminal repeat sequence (ITR) and a left end (LE) minimal ITR sequence, wherein the super minimal piggyBac RE ITR comprises the nucleic acid sequence set forth in any one of SEQ ID NOs: 3-9 and 46-67 and the LE ITR comprises the sequence of SEQ ID NO: 1. In some embodiments, the transposon is a piggyBac transposon or a piggyBac-like transposon.

In some embodiments, the polynucleotide further comprises at least one exogenous nucleic acid sequence. In some embodiments, the at least one exogenous nucleic acid sequence encodes a non-naturally occurring antigen receptor. In some embodiments, the at least one exogenous nucleic acid sequence encodes a therapeutic polypeptide. In some embodiments, the therapeutic polypeptide is polypeptide is Factor VIII polypeptide, Factor IX polypeptide, phenylalanine hydroxylase (PAH), ornithine transcarbamylase (OTC) polypeptide, or methylmalonyl-CoA mutase (MUT1) polypeptide.

In some embodiments, the polynucleotide further comprises a promoter sequence. In some embodiments, the RE ITR is in reverse orientation and/or the LE ITR is in reverse orientation.

In another aspect, provided herein is a vector comprising a polynucleotide described herein.

In another aspect, provided herein is a cell comprising a polynucleotide provided herein.

In another aspect, provided herein is a pharmaceutical composition comprising a cell provided herein and a pharmaceutically acceptable carrier.

In another aspect, provided herein is a transposon, comprising, in 5′ to 3′ order: (i) a left end (LE) inverted terminal repeat (ITR) sequence; (ii) a promoter; (iii) an exogenous nucleic acid sequence encoding a non-naturally occurring antigen receptor; and (iv) a reverse compliment of a super minimal right end (RE) inverted terminal repeat (ITR) sequence.

In another aspect, provided herein is a transposon, comprising, in 5′ to 3′ order: (i) a left end (LE) inverted terminal repeat (ITR) sequence; (ii) a promoter; (iii) an exogenous nucleic acid sequence encoding a therapeutic polypeptide; and (iv) a reverse compliment of a super minimal right end (RE) inverted terminal repeat (ITR) sequence.

In another aspect, provided herein is a transposon, comprising, in 5′ to 3′ order: (i) a right end (RE) inverted terminal repeat (ITR) sequence; (ii) a promoter; (iii) an exogenous nucleic acid sequence encoding a non-naturally occurring antigen receptor; and (iv) a reverse compliment of a super minimal left end (LE) inverted terminal repeat (ITR) sequence.

In another aspect, provided herein is a transposon, comprising, in 5′ to 3′ order: (i) a right end (RE) inverted terminal repeat (ITR) sequence; (ii) a promoter; (iii) an exogenous nucleic acid sequence encoding a therapeutic polypeptide; and (iv) a reverse compliment of a super minimal left end (LE) inverted terminal repeat (ITR) sequence.

In some embodiments, the therapeutic polypeptide is Factor VIII polypeptide, Factor IX polypeptide, phenylalanine hydroxylase (PAH), ornithine transcarbamylase (OTC) polypeptide, or methylmalonyl-CoA mutase (MUT1) polypeptide.

In another aspect, provided herein is a method of treating a disease or disorder in a subject in need thereof comprising administering to the subject (i) at least one therapeutically effective dose of a vector described herein, or a transposon described herein, and (ii) a transposase or a nucleic acid or nucleic acid sequence encoding a transposase enzyme. In some embodiments, the transposase is a SPB transposase, a TAL-ss-SPB PBx transposase fusion protein, or a ZNF-ssSPB transposase fusion protein.

In another aspect, provided herein is a method of treating a disease or disorder in a subject in need thereof comprising administering to the subject at least one therapeutically effective dose of a cell described herein. In some embodiments, the disease or disorder is cancer, a liver disease or disorder, a urea cycle disorder, a metabolic liver disorder or a hemophilia disease.

All documents cited herein, including any cross referenced or related patent or application are hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention.

### DETAILED DESCRIPTION

Provided herein are super minimal PB transposon Inverted Terminal Repeat (ITR) sequences, in particular, super minimal PB transposon right end (RE) ITR polynucleotides, transposons comprising the super minimal PB ITR polynucleotides, and methods of use thereof. It is believed that the minimal ITRs are advantageous because they improve the frequency of transposition. Without wishing to be bound by theory, it is believed that shorter sequence reduces transposon and plasmid size, improving transposion. Moreover, since left and right ITRs are similar in sequence, superminimal ITRs reduce the repetitiveness of the plasmid, which can improve its stability when it is being produced.

Additionally, provided are methods of treating a disease or disorder in a subject in need thereof comprising administering to the subject at least one therapeutically effective dose of a polynucleotide, transposon, vector, cell or composition described herein. In some embodiments, the disease or disorder is cancer, a liver disease or disorder, a urea cycle disorder, a metabolic liver disorder or a hemophilia disease.

### Transposition Systems

### Transposase

Any suitable transposase may be used to introduce the super minimal transposons described herein into a cell. In some embodiments, the transposase is a piggyBac transposase. In some embodiments, the transposase is a super piggyBac (SPB) transposase. In some embodiments, a TAL-ssSPB PBx fusion protein is used, e.g., a TAL-ssPBx fusion protein comprising a TAL array targeting DNA sequences flanking upstream and downstream of a TTAA integration site fused to a piggyBac transposase comprising an N-terminal deletion of amino acids 1-93 and further comprising four hyperactive SPB mutations as well as mutations that render the transposase integration deficient but retains normal excision activity (PBx).

The transposon of the present disclosure can be a piggyBac (PB) transposon. In certain aspects, the transposon comprises a super minimal transposon ITR. In some aspects, the transposon is delivered to a cell using nanotransposon. Nano transposons are described in, e.g., International Patent Application publication No.: WO2020132396, which is incorporated herein by reference in its entirety for examples of nanotransposons that may be used to deliver the transposons described herein to a cell.

In one aspect, provided herein is a polynucleotide encoding a transposon, comprising a super minimal piggyBac right end (RE) inverted terminal repeat sequence (ITR) and a left end (LE) minimal ITR sequence.

It will be apparent to a person of skill in the art that the RE ITR may be located 5′ to the LE ITRs or 3′ to the LE ITR, as long as the 3′ ITR sequence is a reverse complement of the 5′ ITR sequence. Thus, in another aspect, provided herein is a transposon, comprising, in 5′ to 3′ order: (i) a left end (LE) inverted terminal repeat (ITR) sequence; (ii) a promoter; (iii) an exogenous nucleic acid sequence encoding a therapeutic polypeptide or a non-naturally occurring antigen receptor; and (iv) a reverse compliment of a super minimal right end (RE) inverted terminal repeat (ITR) sequence.

In another aspect, provided herein is a transposon, comprising, in 5′ to 3′ order: (i) a right end (RE) inverted terminal repeat (ITR) sequence; (ii) a promoter; (iii) an exogenous nucleic acid sequence encoding a therapeutic polypeptide or a non-naturally occurring antigen receptor; and (iv) a reverse compliment of a super minimal left end (LE) inverted terminal repeat (ITR) sequence.

In certain aspects, the super minimal transposon ITR is a super minimal piggyBac right end (RE) ITR provided herein. In some embodiments, the super minimal piggyBac RE ITR. comprises the nucleic acid sequence set forth in any one of SEQ ID NOs.: 3-9 or 46-67.

In some embodiments, the super minimal RE PB ITR comprises the nucleic acid sequence CCCTAGAAAGATAATCA (SEQ ID NO: 9). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 9. In some embodiments, the super minimal RE PB ITR comprises the nucleic acid sequence CCCTAGAAAGATAATCATA (SEQ ID NO: 8). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 8. In some embodiments, the super minimal RE PB ITR comprises the nucleic acid sequence CCCTAGAAAGATAATCATATTGTG (SEQ ID NO: 7). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 7. In some embodiments, the super minimal RE PB ITR comprises the nucleic acid sequence CCCTAGAAAGATAATCATATTGTGACGTA (SEQ ID NO: 6). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 6. In some embodiments, the super minimal RE PB ITR comprises the nucleic acid sequence CCCTAGAAAGATAATCATATTGTGACGTACGTTA (SEQ ID NO: 5). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 5. In some embodiments, the super minimal RE PB ITR comprises the nucleic acid sequence CCCTAGAAAGATAATCATATTGTGACGTACGTTAAAGAT (SEQ ID NO. 4. In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 4. In some embodiments, the super minimal RE PB ITR comprises the nucleic acid sequence CCCTAGAAAGATAATCATATTGTGACGTACGTTAAAGATAATCA (SEQ ID NO. 3). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 3.

In some embodiments, the super minimal RE PB ITR comprises the nucleic acid sequence CCCTAGAAAGATAATC (SEQ ID NO: 46). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 46.

In some embodiments, the super minimal RE PB ITR comprises the nucleic acid sequence CCCTAGAAAGATAATCAT (SEQ ID NO: 47). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 47.

In some embodiments, the super minimal RE PB ITR comprises the nucleic acid sequence CCCTAGAAAGATAATCATAT (SEQ ID NO: 48). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 48.

In some embodiments, the super minimal RE PB ITR comprises the nucleic acid sequence CCCTAGAAAGATAATCATATT (SEQ ID NO: 49). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 49.

In some embodiments, the super minimal RE PB ITR comprises the nucleic acid sequence CCCTAGAAAGATAATCATATTG (SEQ ID NO: 50). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 50.

In some embodiments, the super minimal RE PB ITR comprises the nucleic acid sequence CCCTAGAAAGATAATCATATTGT (SEQ ID NO: 51). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 51.

In some embodiments, the super minimal RE PB ITR comprises the nucleic acid sequence CCCTAGAAAGATAATCATATTGTGA (SEQ ID NO: 52). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 52.

In some embodiments, the super minimal RE PB ITR comprises the nucleic acid sequence CCCTAGAAAGATAATCATATTGTGAC (SEQ ID NO: 53). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 53.

In some embodiments, the super minimal RE PB ITR comprises the nucleic acid sequence CCCTAGAAAGATAATCATATTGTGACG (SEQ ID NO: 54). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 54.

In some embodiments, the super minimal RE PB ITR comprises the nucleic acid sequence CCCTAGAAAGATAATCATATTGTGACGT (SEQ ID NO: 55). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 55.

In some embodiments, the super minimal RE PB ITR comprises the nucleic acid sequence CCCTAGAAAGATAATCATATTGTGACGTAC (SEQ ID NO: 56). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 56.

In some embodiments, the super minimal RE PB ITR comprises the nucleic acid sequence CCCTAGAAAGATAATCATATTGTGACGTACG (SEQ ID NO: 57). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 57.

In some embodiments, the super minimal RE PB ITR comprises the nucleic acid sequence CCCTAGAAAGATAATCATATTGTGACGTACGT (SEQ ID NO: 58). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 58.

In some embodiments, the super minimal RE PB ITR comprises the nucleic acid sequence CCCTAGAAAGATAATCATATTGTGACGTACGTT (SEQ ID NO: 59). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 59.

In some embodiments, the super minimal RE PB ITR comprises the nucleic acid sequence CCCTAGAAAGATAATCATATTGTGACGTACGTTAA (SEQ ID NO: 60). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 60.

In some embodiments, the super minimal RE PB ITR comprises the nucleic acid sequence CCCTAGAAAGATAATCATATTGTGACGTACGTTAAA (SEQ ID NO: 61). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 61.

In some embodiments, the super minimal RE PB ITR comprises the nucleic acid sequence CCCTAGAAAGATAATCATATTGTGACGTACGTTAAAG (SEQ ID NO: 62). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 62.

In some embodiments, the super minimal RE PB ITR comprises the nucleic acid sequence CCCTAGAAAGATAATCATATTGTGACGTACGTTAAAGA (SEQ ID NO: 63). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 63.

In some embodiments, the super minimal RE PB ITR comprises the nucleic acid sequence CCCTAGAAAGATAATCATATTGTGACGTACGTTAAAGATA (SEQ ID NO: 64).

In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 64.

In some embodiments, the super minimal RE PB ITR comprises the nucleic acid sequence CCCTAGAAAGATAATCATATTGTGACGTACGTTAAAGATAA (SEQ ID NO: 65). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 65.

In some embodiments, the super minimal RE PB ITR comprises the nucleic acid sequence CCCTAGAAAGATAATCATATTGTGACGTACGTTAAAGATAAT (SEQ ID NO: 66). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 66.

In some embodiments, the super minimal RE PB ITR comprises the nucleic acid sequence CCCTAGAAAGATAATCATATTGTGACGTACGTTAAAGATAATC (SEQ ID NO: 67). In some embodiments, the super minimal RE PB ITR comprises a nucleic acid sequence that is at least 75%, at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO: 67.

In some embodiments, the transposon further comprises a piggyBac left end (LE) minimal ITR sequence comprising the nucleic acid sequence of SEQ ID NO: 1).

In some aspects when the transposon is a PB transposon, the transposase is a piggyBac® (PB) transposase, a piggyBac-like (PBL) transposase or a Super piggyBac® (SPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296, each of which is incorporated herein by reference in its entirety for examples of transposases that may be used in conjunction with the transposons described herein.

Without wishing to be bound by theory, it is believed that the PB, PBL and SPB transposases recognize transposon-specific inverted terminal repeat sequences (ITRs) on the ends of the transposon, and then insert the nucleotide sequence located between the ITRs of the transposons at a target site. The target sequence of the PB or PBL transposon can comprise or consist of the sequence 5′-TTAT-3′, 5′-TTAA-3′,5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ or 5′-TTTT-3′. In some embodiments, the PB or PBL transposon system has no size limit for the genes of interest that can be included between the ITRs.

Illustrative amino acid sequence for one or more PB, PBL and SPB transposases are disclosed in U.S. Pat. Nos. 6,218,185; 6,962,810 and 8,399,643, each of which is incorporated herein by reference in its entirety for examples of transposases that may be used in conjunction with the transposons described herein.

The PB or PBL transposase can comprise or consist of an amino acid sequence having an amino acid substitution at two or more, at three or more or at each of positions 30, 165, 226, 282, or 538 of the sequence of SEQ ID NO: 45 (with numbering beginning at the 12th amino acid of SEQ ID NO: 45). In some embodiment, the amino acid substitutions are hyperactivity amino acid substitutions. The transposase can be a SPB transposase that comprises or consists of the amino acid sequence of the sequence of SEQ ID NO: 45 with one, two, three, for or all of the hyperactivity amino acid substitutions 130V, G165S, M226F, M282V, and N538K, with the numbering beginning at position 12 of SEQ ID NO: 45. In an aspect, the SPB transposase comprises an amino acid sequence at least 75%, at least 80%, at least 85%, at least 90%, at least 95%, at least 96%, at least 97%, at least 98%, or at least 99% identical to the amino acid sequence set forth in SEQ ID NO: 45. In some embodiments, the SPB transposase comprises the amino acid sequence set forth in SEQ ID NO: 45 with one, two, three, four or five conversative amino acid substitutions. In some embodiments, the SPB transposase comprises the amino acid sequence set forth in SEQ ID NO: 45.

### Formulations, Dosages and Modes of Administration

The present disclosure provides formulations, dosages and methods for administration of the compositions and cells described herein. In one aspect, provided herein is a pharmaceutical composition comprising a tandem dimer transposase described herein and a pharmaceutically acceptable carrier. In another aspect, provided herein is a pharmaceutical composition comprising a modified cell described herein and a pharmaceutically acceptable carrier.

The disclosed compositions and pharmaceutical compositions can comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means. In preferred embodiments, a composition comprising a modified cell described herein is administered intravenously, e.g., by intravenous infusion.

A composition of the disclosure can be prepared for use for parenteral (subcutaneous, intramuscular or intravenous) or any other administration particularly in the form of liquid solutions or suspensions. For parenteral administration, a composition disclosed herein can be formulated as a solution, suspension, emulsion, particle, powder, or lyophilized powder in association, or separately provided, with a pharmaceutically acceptable parenteral vehicle.

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection or infusion can be a non-toxic, non-orally administrable diluting agent, such as aqueous solution, a sterile injectable solution or suspension in a solvent.

As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like.

Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Methods of Treatment

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

In some aspects, the treatment of a disease or disorder comprises adoptive cell therapy. For example, in an aspect, the disclosure provides modified cells that express a chimeric antigen receptor (CAR). The transposons described herein may be used to generate such modified cells. The modified cells may be allogeneic or autologous to the patient. In some preferred embodiments, the modified cell is an allogeneic cell. In some embodiments, the modified cell is an autologous T-cell or a modified autologous CAR T-cell. In some preferred embodiments, the modified cell is an allogeneic T-cell or a modified allogeneic CAR T-cell.

In some embodiments, the disease or disorder treated in accordance with the methods described herein is a cancer. Non-limiting examples of cancer includes leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In another non-limiting example, the present disclosure provides methods of treating a metabolic liver disorder in a subject, the methods comprising administering to the subject: a) at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the metabolic liver disorder can be Ornithine Transcarbamylase (OTC) Deficiency and the at least one therapeutic protein can comprise ornithine transcarbamylase (OTC) polypeptide. In some aspects, the metabolic liver disorder can be methylmalonic acidemia (MMA) and the at least one therapeutic protein can comprise a methylmalonyl-CoA mutase (MUT1) polypeptide.

In a non-limiting example, the present disclosure provides methods of treating a hemophilia disease in a subject, the methods comprising administering to the subject: at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the hemophilia disease can be hemophilia A and the at least one therapeutic protein can comprise Factor VIII. In some aspects, the hemophilia disease can be hemophilia B and the at least one therapeutic protein can comprise Factor IX.

In a non-limiting example, the present disclosure provides methods of treating phenylketonuria (PKU) in a subject, the methods comprising administering to the subject: at least one therapeutically effective amount of at least one composition comprising a transposon of the present disclosure comprising a sequence encoding the phenylalanine hydroxylase gene; and b) at least one therapeutically effective amount of a composition comprising a nucleic acid sequence encoding at least one transposase.

In a non-limiting example, the present disclosure provides for methods of treating a disease or disorder in a subject by administering to the subject in need thereof a therapeutically effective amount of an LNP composition comprising a DNA transposon encoding a therapeutic protein comprising a super minimal ITR and an mRNA encoding a piggyBac transposase. In some embodiments, the disease or disorder is cancer, a liver disease or disorder, a urea cycle disorder, a metabolic liver disorder or a hemophilia disease.

In a non-limiting example, the present disclosure provides for methods of treating a disease or disorder in a subject by administering to the subject in need thereof a therapeutically effective amount of a first LNP composition comprising a DNA transposon encoding a therapeutic protein comprising a super minimal ITR, and a second LNP composition comprising an mRNA encoding a piggyBac transposase. In some embodiments, the disease or disorder is cancer, a liver disease or disorder, a urea cycle disorder, a metabolic liver disorder or a hemophilia disease. In some embodiments, the disease or disorder is an autoimmune disease. In one embodiment, the autoimmune disease is autoimmune neutropenia, Guillain-Barré syndrome, epilepsy, autoimmune encephalitis, Isaacs' syndrome, nevus syndrome, pemphigus vulgaris, deciduous pemphigus, bullous pemphigoid, acquired epidermolysis bullosa, gestational pemphigoid, mucous membrane pemphigoid, antiphospholipid syndrome, autoimmune anemia, myasthenia gravis, autoimmune Graves' disease, thyroid eye disease (TED), Goodpasture syndrome, multiple sclerosis, rheumatoid arthritis, lupus, idiopathic thrombocytopenia purpura (ITP), warm autoimmune hemolytic anemia (WAIHA), chronic inflammatory demyelinating polyneuropathy (CIDP), lupus nephritis, or membranous nephropathy.

The dosage of a pharmaceutical composition to be administered to a subject can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired.

In aspects where the compositions to be administered to a subject in need thereof are modified cells as disclosed herein, between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered. In some embodiments, the cells are administered at a dose of between about 5×10⁶ and about 25×10⁶ cells.

In other embodiments, the dosage of cells may depend on the body weight of the person, e.g., between about 1×10³ and about 1×10⁴ cells; between about 1×10⁴ and about 1×10⁵ cells; between about 1×10⁵ and about 1×10⁶ cells; between about 1×10⁶ and about 1×10⁷ cells; between about 1×10⁷ and about 1×10⁸ cells; between about 1×10⁸ and about 1×10⁹ cells; between about 1×10⁹ and about 1×10¹⁰ cells, between about 1×10¹⁰ and about 1×10¹¹ cells, between about 1×10¹¹ and about 1×10¹² cells, between about 1×10¹² and about 1×10¹³ cells, between about 1×10¹³ and about 1×10¹⁴ cells, between about 1×10¹⁴ and about 1×10¹⁵ cells, between about 1×10¹⁵ and about 1×10¹⁶ cells, between about 1×10¹⁶ and about 1×10¹⁷ cells, between about 1×10¹⁷ and about 1×10¹⁸ cells, between about 1×10¹⁸ and about 1×10¹⁹ cells; or between about 1×10¹⁹ and about 1×10²⁰ cells may be administered per kg body weight of the subject.

A more detailed description of pharmaceutically acceptable excipients, formulations, dosages and methods of administration of the disclosed compositions and pharmaceutical compositions is disclosed in PCT Publication No. WO 2019/049816, which is incorporated herein by reference in its entirety.

### Kits

In another aspect, provided herein is a kit comprising a cell line which has been engineered to comprise a modified target site for an SPB or a PBx provided herein within its genome, preferably in a highly expressed genomic region. The kit may further comprise a composition comprising one or more super minimal PB transposon Inverted Terminal Repeat (ITR) sequences described herein. In some embodiments, the cell line is a T cell line.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value.

Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers.

“Consisting of” shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Table 1, Table 2, and Table 3 below. In some aspects, conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Illustrative conservative substitutions are set out in Table 1.

TABLE 1
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 2.

TABLE 2
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, illustrative conservative substitutions are set out in Table 3.

TABLE 3
Conservative Substitutions III

| Original Residue | Illustrative Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

In certain embodiments, if a sequence has a certain sequence identity (e.g., 75%, 80%, 85%, 90%, 95%, 98%, or 99%) to a certain SEQ ID NO, the sequence and the sequence of the SEQ ID NO have the same length. In certain embodiments, if a sequence has a certain sequence identity (e.g., 75%, 80%, 85%, 90%, 95%, 98%, or 99%) to a certain SEQ ID NO, the sequence and the sequence of the SEQ ID NO only differ due to conservative amino acid substitutions.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

The Examples in this section are provided for illustration and are not intended to limit the invention.

### Example 1: Construction of Super Minimal PiggyBac Inverted Terminal Repeat Polynucleotides

A set of truncated, super minimal PB RE ITR polynucleotides of the piggy Bac 63 bp minimal right end (RE) ITR (SEQ ID NO: 2) was constructed by sequential deletion of the 3′ end of the minimal RE ITR sequence to generate super minimal piggyBac RE ITR variants. The super minimal RE ITR variants comprise the first 17 bp, 19 bp, 24 bp, 29 bp, 34 bp, 39 bp, or 44 bp of the 63 bp minimal RE ITR sequence set forth in SEQ ID NO: 2, resulting in nucleic acid sequences of SEQ ID NOs: 3-9, respectively.

Briefly, the nucleic acids encoding the super minimal RE ITR variants were synthesized, purified and used to generate transposons comprising the super minimal RE ITR variants.

### Example 2: Effect of Super Minimal PB RE ITR Polynucleotide Length on Super PiggyBac (SPB) Transposase Excision Activity

The super minimal PB ITR polynucleotides prepared in Example 1 were initially tested using a luciferase transposon excision reporter. Briefly, the excision reporter contains a PGK promoter (SEQ ID NO: 10) followed by a disrupted NanoLuc® luciferase coding sequence followed by a SV40 late poly adenylation signal sequence (SEQ ID NO: 11). The NanoLuc® sequence is disrupted at a TTAA such that the 5′ end of NanoLuc® (SEQ ID NO: 12) is separated from the 3′ end of NanoLuc® (SEQ ID NO: 13) by a PB transposon. The transposon from 5′ to 3′ comprises a TTAA sequence, the 35 bp LE ITR (SEQ ID NO: 1), a 189 bp “cargo” (SEQ ID NO: 14), the reverse complement of the 63 bp RE (SEQ ID NO: 2) or a truncated RE ITR comprising one of SEQ ID NOs: 3-9, and a TTAA sequence. The reporter constructs were co-transfected into cells along with a plasmid expressing a Super piggyBac (SPB) transposase. If the super minimal ITRs are functional, excision of the transposon comprising the super minimal PB RE ITRs and seamless repair of the reporter coding sequence results in NanoLuc® expression that can be detected through a luciferase assay.

Briefly, on Day 0, 30,000 HEK293T cells were reverse transfected using 20 ng of the reporter constructs, 10 ng of a SPB expression vector, and 20 ng of carrier DNA using 0.15 μL of Transit2020 transfection reagent in accordance with the manufacturer's instructions, and the cells were plated in 100 μL of DMEM medium+10% FBS in 96 well plates. The cells were incubated at 37° C. and the luciferase activity was measured on Day 1. The results are shown in Table 1.

TABLE 1

| | Luciferase Signal | | |
|---|---|---|---|
| | Replicate 1 | Replicate 2 | Average |
| Minimal ITR | 650235 | 713407 | 681821 |
| 44 bp Super Minimal ITR | 797485 | 762120 | 779803 |
| 39 bp Super Minimal ITR | 538296 | 542674 | 540485 |
| 34 bp Super Minimal ITR | 681352 | 688176 | 684764 |
| 29 bp Super Minimal ITR | 950922 | 958638 | 954780 |
| 24 bp Super Minimal ITR | 491079 | 505393 | 498236 |
| 19 bp Super Minimal ITR | 1362175 | 1526605 | 1444390 |
| 17 bp Super Minimal ITR | 1018028 | 1025618 | 1021823 |

As shown in Table 1, luciferase activity was detected in transposed cells for all super minimal PB RE ITR variants with the highest signal observed using the super minimal 19 bp PB RE ITR.

In a second experiment, the reporter constructs comprising the 17 bp PB RE super minimal ITR or the 19 bp PB RE super minimal ITR were tested in the luciferase excision assay as well as a reporter construct comprising Full PB ITRs comprising an intact PB 5′UTR sequence (SEQ ID NO: 15) and an intact 3′UTR sequence (SEQ ID NO: 16) as well as a reporter construct comprising minimal PB ITRs comprising the 35 bp LE ITR (SEQ ID NO: 1) and the 63 bp RE (SEQ ID NO: 2). The results are shown in Table 2.

TABLE 2

| | Luciferase Signal | | | |
|---|---|---|---|---|
| | Replicate 1 | Replicate 2 | Replicate 3 | Average |
| Minimal ITR | 612325 | 483668 | 614112 | 570035 |
| 17 bp Super | 744409 | 755379 | 908710 | 802833 |
| Minimal ITR | | | | |
| 19 bp Super | 1101035 | 1190137 | 1256866 | 1182679 |
| Minimal ITR | | | | |
| Full ITR | 382459 | 453890 | 415051 | 417133 |

As shown in Table 2, the 19 bp PB RE super minimal ITR reporter outperformed the minimal 63 bp RE ITR as well as a Full PB ITR reporter that further comprised the PB 5′UTR and 3′ UTR added back to the PB LE ITR and super minimal PB RE ITR, respectively.

### Example 3: Effect of Transposon DNA Cargo Size on SPB Transposase Integration and Excision Activity of Transposons Comprising Super Minimal PB RE ITRs

A dual excision/integration luciferase reporter system was used to test full ITRs, minimal ITRs and super minimal ITRs effect on SPB transposase activity of integrating or excising a larger transposon in comparison of the transposon of Example 2. The reporter system comprises a firefly luciferase open reading frame disrupted by a SPB transposon. Initially, firefly luciferase is not expressed, but SPB-mediated excision of the transposon and seamless repair results in expression. The transposon itself expresses a destabilized NanoLuc® luciferase mRNA. NanoLuc® expression from the episomal vector is unstable, since the mRNA lacks a polyA tail and contains 3′ destabilization element. Integration of the transposon into genomic DNA allows the mRNA to utilize a genomic polyA sequence and splice out the destabilization element using a splice donor sequence on the transposon, leading to luciferase expression (SEQ ID NO: 17).

The transposon ITRs of this reporter plasmid were modified to prepare transposons that comprise: full ITRs, minimal ITRs or 19 bp RE super minimal ITRs. Briefly, on Day 0, about 200,000 K562 cells were nucleofected using 50 ng of one of the reporters and 500 ng of a SPB expression vector (or carrier DNA as a negative control) and plated in 600 μL of IMDM medium+10% FBS in 24 well plates. The cells were incubated at 37° C., and the luciferase activity was measured on Day 2. The results are shown in Table 3.

TABLE 3

| | +Transposase | | | | No Transposase | | | |
|---|---|---|---|---|---|---|---|---|
| | Rep. 1 | Rep. 2 | Rep. 3 | Average | Rep. 1 | Rep. 2 | Rep. 3 | Average |
| | Firefly Luciferase Excision Signal | | | | | | | |
| Full ITR | 9260 | 7918 | 8774 | 8651 | 103 | 85 | 161 | 116 |
| Minimal | 5379 | 4874 | 4388 | 4880 | 277 | 147 | 196 | 207 |
| ITR | | | | | | | | |
| Super | 1599 | 1878 | 1283 | 1587 | 226 | 108 | 208 | 181 |
| Minimal | | | | | | | | |
| ITR | | | | | | | | |
| No | 221 | 250 | 374 | 282 | 101 | 92 | 214 | 136 |
| Transposon | | | | | | | | |
| | NanoLuc ® Luciferase Excision Signal | | | | | | | |
| Full ITR | 1139309 | 1090008 | 1068629 | 1099315 | 9687 | 12020 | 12125 | 11277 |
| Minimal | 598288 | 595721 | 677798 | 623936 | 37532 | 41317 | 45237 | 41362 |
| ITR | | | | | | | | |
| Super | 98109 | 109296 | 115920 | 107775 | 43946 | 41632 | 46487 | 44022 |
| Minimal | | | | | | | | |
| ITR | | | | | | | | |
| No | 355 | 91 | 100 | 182 | 307 | 328 | 186 | 274 |
| Transposon | | | | | | | | |

Rep.: Replicate.

As shown in Table 3, the detected firefly luciferase excision signal and the NanoLuc® integration signals were each highest for the full PB ITRs, lower for the minimal PB ITRs, and lowest for the 19 bp PB RE super minimal ITRs. Transposons comprising larger DNA cargo result in the placement of the LE ITR and RE ITR farther apart, suggesting lack of a second transposase dimer binding site in the super minimal RE ITR may reduce transposition efficiency for larger DNA cargos.

### Example 4: SPB Transposase Integration and Excision Activity of Spacer Length Between PB LE ITR and PB RE ITR of Transposons Comprising Super Minimal PB RE ITRs

Transposons typically are delivered to cells, ex vivo or in vivo, on circular DNA vectors. A strategy to bring ITRs in closer proximity on circular vectors, even for large transposons, is to reduce the number of base pairs separating the ITRs outside of the transposon (i.e., the plasmid backbone).

The dual luciferase reporters prepared in Example 3 were modified to place the ITRs close together, essentially converting the entire vector into a transposon, to generate transposons comprising Full PB ITRs (SEQ ID NO: 18), minimal PB ITRs (SEQ ID NO: 19) or super minimal PB ITRs (SEQ ID NO: 20) and a 221 bp sequence between the TTAA's outside of the transposon. The firefly excision reporter was partially deleted in the process but the NanoLuc® integration reporter remained intact. These new integration-only luciferase reporters were tested for transposon integration in K562 cells as described in Example 3 along with a positive control luciferase reporter comprising a full-length plasmid backbone. The results are shown in Table 4.

TABLE 4

| | Luciferase Signal | | | | | |
|---|---|---|---|---|---|---|
| | +Transposase | | | No Transposase | | |
| | Replicate 1 | Replicate 2 | Average | Replicate 1 | Replicate 2 | Average |
| Far Apart Full | 459720 | 448870 | 454295 | 13733 | 13335 | 13534 |
| ITR | | | | | | |
| Adjacent Full | 1437067 | 1496970 | 1467019 | 9120 | 9445 | 9283 |
| ITR | | | | | | |
| Adjacent | 2097877 | 1734855 | 1916366 | 3072 | 2634 | 2853 |
| Minimal ITR | | | | | | |
| Adjacent | 359589 | 418898 | 389244 | 2592 | 3428 | 3010 |
| Super | | | | | | |
| Minimal ITR | | | | | | |

As shown in Table 4, the full ITRs and minimal ITRs resulted in high transposition, while lower levels of transposition were observed using the 19 bp super minimal ITRs suggesting that super minimal ITRs work best for SPB-mediated transposition of very small transposons.

### Example 5: Effect of Super Minimal PB RE ITR Polynucleotide Length on Site-Specific, Excision Only, TAL-Super PiggyBac Fusion Proteins (TAL-Ss-SPB PBx) on Transposase Excision Activity

The NanoLuc® excision only reporter system described in Example 2 was employed to compare the excision activities of reporter plasmids comprising transposons comprising Full ITRs, minimal ITRs, or the 44 bp, 34 bp, 19 bp, and 17 bp RE super minimal ITR variants. Briefly, 30,000 HEK293T cells were reverse transfected as described in Example 2 with a GFP left targeting TAL-ssSPB PBx expression vector (SEQ ID NO: 21) and with a GFP right targeting TAL-ssSPB PBx expression vector (SEQ ID NO: 22) along with each reporter. The GFP targeting TAL-ssSPB PBx expression vectors comprises a TAL array targeting DNA sequences flanking upstream and downstream of a TTAA integration site fused to a piggyBac transposase comprising an N-terminal deletion of amino acids 1-93 and further comprising four hyperactive SPB mutations as well as mutations that render the transposase integration deficient but retains normal excision activity (PBx). TAL Array-SPB transposase fusion proteins GFP1 Right TAL-ssSPB PBx and GFP1 Left TAL-ssSPB PBx targeted to specific, 10 bp right and 10 bp left sequences in the coding region of the GFP gene were prepared as described in Examples 14 and 18 of co-owned International Patent Application Publication No. PCT/2022/22549, the contents of which are incorporated by reference in its entirety.

Luciferase signals were measured from cells transfected with transposon comprising the various ITR sequences. The results are shown in Table 5.

TABLE 5

| | Luciferase Signal | | | | | |
|---|---|---|---|---|---|---|
| | GFP1L TAL-ssSPB | | | GFP1R TAL-ssSPB | | |
| | Replicate 1 | Replicate 2 | Average | Replicate 1 | Replicate 2 | Average |
| Full ITR | 2556812 | 1997313 | 2277063 | 2892092 | 2532898 | 2712495 |
| Minimal ITR | 3050627 | 2562406 | 2806517 | 2260078 | 1918567 | 2089323 |
| 44 bp Super | 2790164 | 2137331 | 2463748 | 3021131 | 2352675 | 2686903 |
| Minimal ITR | | | | | | |
| 34 bp Super | 4283716 | 2900951 | 3592334 | 4777177 | 3704723 | 4240950 |
| Minimal ITR | | | | | | |
| 19 bp Super | 4617430 | 3482804 | 4050117 | 4724253 | 3935542 | 4329898 |
| Minimal ITR | | | | | | |
| 17 bp Super | 4477759 | 3212069 | 3844914 | 5073659 | 4371134 | 4722397 |
| Minimal ITR | | | | | | |

As shown in Table 5, transfected cells comprising any of the reporter constructs resulted in production of a luciferase signal, indicating transposon excision from the reporter and restoration of a full-length luciferase coding sequence. The transposons comprising Full ITRs, the minimal ITRs, or the 44 bp super minimal ITR (which retains a portion of the distal transposase binding site) all performed similarly. Transposons comprising the 34 bp, 19 bp, and 17 bp super minimal ITRs, which all have completely deleted the binding site for the distal transposase dimer, each exhibited higher excision signals than transposons comprising Full ITRs or larger ITR variants thereof.

### Example 6: Effect of Super Minimal PB RE ITR Polynucleotide Length on Site-Specific, Excision Only, TAL-Super PiggyBac Fusion Proteins (TAL-Ss-SPB PBx) on Transposase Excision Activity

Polynucleotides comprising Full ITRs, minimal ITRs, or the 19 bp super minimal ITRs were examined to accommodate transposition of a large transposon using the dual excision/integration luciferase reporter and SPB transposase and TAL-ssSPB PBx fusion proteins by transfecting HEK293T as described above in Example 3. Only the excision signal of the dual excision/integration luciferase reporter was monitored in view of the TAL-ssSPB consists of excision only PBx transposase delta 1-93 sequence and the targeted integration sites for the GFP TAL-ssSPBs are not present in the genome. The results of the excision assay are shown in Table 6.

TABLE 6

| | Luciferase Signal | | |
|---|---|---|---|
| | | | Super Minimal |
| | Full ITR | Minimal ITR | ITR |
| GFP1L TAL-ssSPB | 6784 | 6421 | 7414 |
| GFP1R TAL-ssSPB | 6585 | 7885 | 10375 |
| SPB | 11052 | 7260 | 3997 |
| Catalytic Dead SPB | 486 | 468 | 334 |

As shown in Table 6, the highest excision activity was observed for SPB transposase using reporter constructs comprising full ITRs, with less excision activity using minimal ITRs, and the lowest excision activity using super minimal ITRs. This trend, however, was partly reversed using the TAL-ssSPB PBx fusion proteins where reporter constructs comprising super minimal ITRs exhibited the highest excision activity compared to reporter constructs comprising Full ITRs or minimal ITRs, which performed similarly but to a lesser extent. As a negative control for the assay, catalytically dead SPB exhibited background levels of luciferase activity.

### Example 7: Construction of TAL Binding ITRs and Use with TAL-ssSPB PBx Fusion Proteins for Site Specific Transposition

A series of super minimal RE ITR variants were constructed by extending the super minimal RE ITR sequence using spacers of various lengths followed by the reporter plasmid target site for the second distal GFP1 R TAL-ssSPB, which includes the TAL binding site (SEQ ID NO: 23), a spacer, then the binding sites for the PBx DBD and CRD domain. Several TAL RE ITR variants of differing spacer length between there RE ITR and the TAL Array (“TAL Binding RE ITRs”) were constructed: no spacer (SEQ ID NO: 24); a +5 bp spacer (SEQ ID NO: 25), a +10 bp spacer (SEQ ID NO: 26); a +15 bp spacer (SEQ ID NO: 27), a +20 bp spacer (SEQ ID NO: 28) and a +25 bp spacer (SEQ ID NO: 29). Increasing spacer length further separates the binding site for the first proximal left TAL-ssSPB PBx dimer and the second distal right TAL-ssSPB PBx dimer.

Analogously, a series of super minimal LE ITR variants were constructed for the second distal TAL-ssSPB dimer. Several TAL LE ITR variants of differing spacer length between the LE ITR and the TAL Array (“TAL Binding LE ITRs”) were constructed: no spacer (SEQ ID NO: 30); a +5 bp spacer (SEQ ID NO: 31), a +10 bp spacer (SEQ ID NO: 32); a +15 bp spacer (SEQ ID NO: 33), a +20 bp spacer (SEQ ID NO: 34) and a +25 bp spacer (SEQ ID NO: 35). To avoid potential recombination between these extended super minimal LE and RE ITRs, the spacer sequences for the LE and RE ITR pairs were modified to reduce sequence repetitiveness and homology.

The TAL binding LE and RE ITRs were initially tested using the luciferase transposon excision reporter in HEK293T as described above in Example 2. Each reporter plasmid was co-transfected with either the GFP1 R TAL-ssSPB PBx (SEQ ID NO: 22), which binds the ITRs, or with the PAH2 L TAL-ssSPB PBx (SEQ ID NO: 36), which does not bind the ITRs. PAH2 L TAL-ssSPB PBx was prepared as described in co-owned International Patent Application Publication No. PCT/2022/22549. The GFP1 R TAL-ssSPB PBx resulted in higher excision signal with all the TAL binding ITR reporters than the PAH2 L TAL ssSPB PBx. As controls, reporter constructs comprising minimal and super minimal ITRs were transfected, in which case both TAL-ssSPBs PBx fusion proteins resulted in similar excision signal. The results are shown in Table 7.

TABLE 7

| | Luciferase Signal | | | | | |
|---|---|---|---|---|---|---|
| | GFP1R TAL-ssSPB PBx | | | PAH2L TAL-ssSPB PBx | | |
| | Replicate | Replicate | | Replicate | Replicate | |
| | 1 | 2 | Average | 1 | 2 | Average |
| TAL Binding | 9921443 | 9129146 | 9525295 | 5494391 | 5729109 | 5611750 |
| ITR + 25 bp | | | | | | |
| TAL Binding | 8079110 | 8537733 | 8308422 | 5506535 | 5243049 | 5374792 |
| ITR + 20 bp | | | | | | |
| TAL Binding | 7350781 | 8428584 | 7889683 | 6011447 | 5671538 | 5841493 |
| ITR + 15 bp | | | | | | |
| TAL Binding | 5787803 | 6541725 | 6164764 | 5393845 | 5496607 | 5445226 |
| ITR + 10 bp | | | | | | |
| TAL Binding | 9586499 | 8519096 | 9052798 | 6699984 | 6224914 | 6462449 |
| ITR + 5 bp | | | | | | |
| TAL Binding | 6630131 | 6208129 | 6419130 | 6017608 | 6026622 | 6022115 |
| ITR | | | | | | |
| Super | 11369627 | 11068956 | 11219292 | 11646813 | 11118015 | 11382414 |
| Minimal ITR | | | | | | |
| Minimal ITR | 3623991 | 3607151 | 3615571 | 4549196 | 3866228 | 4207712 |

As shown in Table 7, the highest signal using the TAL binding ITRs was seen with the version containing the longest 25 bp spacer sequence.

### Example 8: Construction of TAL Binding ITRs Targeting LINE1 Elements and Use with LINE1-targeting TAL-ssSPB PBx Fusion Proteins for Site Specific Transposition

### A. Small Transposons

In the first experiment, the various TAL Binding ITR designs were compared for site-specific transposition of small transposons into the human genome at a target site found in LINE1 repeats (SEQ ID NO: 37) catalyzed by LINE1 TAL-ssSPB comprising LINE L1 ss-SPB PBx (SEQ ID NO: 38) and LINE R1 ss-SPB PBx (SEQ ID NO: 39). LINE1 L1 TAL-ssSPB PBx and LINE R1 ss-SPB PBx were prepared as described in co-owned International Patent Application Publication No. PCT/2022/22549. A 610 bp transposon with full ITRs (SEQ ID NO: 40), a 392 bp transposon with TAL binding ITRs with 25 bp spacer (SEQ ID NO: 41), a 365 bp transposon with minimal ITRs (SEQ ID NO: 42), and a 321 bp transposon with 19 bp super minimal ITRs (SEQ ID NO: 43) were cloned into a 4.5 kb donor vector. 450 ng of each transposon donor was co-transfected into 120,000 HEK293T cells (plated one day earlier) along with 50 total ng of the pair of LINE1 TAL-ssSPB pair using 1 μL of JetPrime transfection reagent in accordance with the manufacturer's instructions. Two days later, the genomic DNA was harvested, and site-specific integration of the transposon into the target sites in both the forward and reverse orientations was quantified by ddPCR. The results are shown in Table 8.

TABLE 8

Integrations per Haploid Genome

| | Forward Integration | Reverse Integration |
|---|---|---|
| Full ITR | 2.82 | 2.82 |
| TAL Binding ITR | 4.89 | 5.05 |
| Minimal ITR | 4.25 | 4.04 |
| Super Minimal ITR | 5.68 | 5.03 |

As shown in Table 8, the super minimal ITR transposon resulted in the highest level of site-specific integration of the four ITR designs tested; however, the TAL Binding ITR demonstrated good activity at LINE1 elements.

### B. Large Transposons

In a second experiment, the various ITR designs were next compared for site-specific transposition of large transposons into the human genome at a target site found in LINE1 repeats (SEQ ID NO: 37) catalyzed by LINE1 TAL-ssSPB comprising LINE L1 ss-SPB PBx (SEQ ID NO: 38) and LINE R1 ss-SPB PBx (SEQ ID NO: 39). The transposon donor nanoplasmid contained a PiggyBac transposon containing from 5′ to 3′ direction: TTAA, a 309 bp fragment containing the PiggyBac 5′ ITR and part of the UTR, a “cargo” consisting of an EF1a promoter, a puromycin resistance gene, a 2A peptide, and a GFP reporter, followed by a 238 bp fragment containing the PiggyBac 3′ ITR and part of the UTR, and TTAA (SEQ ID NO: 44). The donor DNA transposon was modified to replace the full ITRs with TAL binding ITRs with 25 bp spacer, minimal ITRs, or 19 bp super minimal ITRs. Each transposon donor was co-transfected into HEK293T cells along with the LINE1 TAL-ssSPB expression vectors. One or three days later, the genomic DNA was harvested and site-specific integration of the transposon into the target sites in the forward orientation was quantified by ddPCR. The results are shown in Table 9.

TABLE 9

| | Integrations per Haploid Genome | | | | | | | |
|---|---|---|---|---|---|---|---|---|
| | Day 1 | | | | Day 3 | | | |
| | Rep 1 | Rep 2 | Rep 3 | Average | Rep 1 | Rep 2 | Rep 3 | Average |
| Full ITR | 0.21 | 0.18 | 0.18 | 0.19 | 0.83 | 1.1 | 1.09 | 1.01 |
| TAL Binding | 0.24 | 0.24 | 0.26 | 0.25 | 0.97 | 1.41 | 1.18 | 1.19 |
| ITR | | | | | | | | |
| Minimal ITR | 0.24 | 0.22 | 0.41 | 0.29 | 0.78 | 0.88 | 1.07 | 0.91 |
| Super | 0.52 | 0.52 | 0.54 | 0.53 | 1.34 | 2.05 | 1.9 | 1.76 |
| Minimal ITR | | | | | | | | |

As shown in Table 9, similar to the first experiment using small transposons, the super minimal ITR transposon resulted in the highest level of site-specific integration of large transposons into LINE1 element repeats.

We claim:

1. A polynucleotide encoding a transposon, comprising a super minimal piggyBac right end (RE) inverted terminal repeat sequence (ITR) and a left end (LE) minimal ITR sequence, wherein the super minimal piggyBac RE ITR comprises the nucleic acid sequence set forth in SEQ ID NO: 8 and the LE ITR comprises the sequence of SEQ ID NO: 1.

2. The polynucleotide of claim 1, wherein the transposon is a piggyBac transposon or a piggyBac-like transposon.

3. The polynucleotide of claim 1 or 2, wherein the polynucleotide further comprises at least one exogenous nucleic acid sequence.

4. The polynucleotide of claim 3, wherein at least one exogenous nucleic acid sequence encodes a non-naturally occurring antigen receptor.

5. The polynucleotide of claim 3, wherein the at least one exogenous nucleic acid sequence encodes a therapeutic polypeptide.

6. The polynucleotide of claim 5, wherein the therapeutic polypeptide is polypeptide is Factor VIII polypeptide, Factor IX polypeptide, phenylalanine hydroxylase (PAH), ornithine transcarbamylase (OTC) polypeptide, or methylmalonyl-CoA mutase (MUT1) polypeptide.

7. The polynucleotide of any one of claims 3-6, further comprising a promoter sequence.

8. The polynucleotide of any one of claims 1-7, wherein the RE ITR is in reverse orientation and/or the LE ITR is in reverse orientation.

9. A vector comprising the polynucleotide of any one of claims 1-8.

10. A cell comprising the polynucleotide of any one of claims 1-8 or the vector of claim 9.

11. A pharmaceutical composition comprising the cell of claim 10, and a pharmaceutically acceptable carrier.

12. A transposon, comprising, in 5′ to 3′ order: (i) a left end (LE) inverted terminal repeat (ITR) sequence; (ii) a promoter; (iii) an exogenous nucleic acid sequence encoding a non-naturally occurring antigen receptor; and (iv) a reverse compliment of a super minimal right end (RE) inverted terminal repeat (ITR) sequence.

13. A transposon, comprising, in 5′ to 3′ order: (i) a left end (LE) inverted terminal repeat (ITR) sequence; (ii) a promoter; (iii) an exogenous nucleic acid sequence encoding a therapeutic polypeptide; and (iv) a reverse compliment of a super minimal right end (RE) inverted terminal repeat (ITR) sequence.

14. A transposon, comprising, in 5′ to 3′ order: (i) a right end (RE) inverted terminal repeat (ITR) sequence; (ii) a promoter; (iii) an exogenous nucleic acid sequence encoding a non-naturally occurring antigen receptor; and (iv) a reverse compliment of a super minimal left end (LE) inverted terminal repeat (ITR) sequence.

15. A transposon, comprising, in 5′ to 3′ order: (i) a right end (RE) inverted terminal repeat (ITR) sequence; (ii) a promoter; (iii) an exogenous nucleic acid sequence encoding a therapeutic polypeptide; and (iv) a reverse compliment of a super minimal left end (LE) inverted terminal repeat (ITR) sequence.

16. The transposon of claim 13 or 15, wherein the therapeutic polypeptide is Factor VIII polypeptide, Factor IX polypeptide, phenylalanine hydroxylase (PAH), ornithine transcarbamylase (OTC) polypeptide, or methylmalonyl-CoA mutase (MUT1) polypeptide.

17. A method of treating a disease or disorder in a subject in need thereof comprising administering to the subject (i) at least one therapeutically effective dose of the vector of claim 9, or the transposon of any one of claims 13-15, and (ii) a transposase or a nucleic acid or nucleic acid sequence encoding a transposase enzyme.

18. The method of claim 17, wherein the transposase is a SPB transposase, a TAL-ss-SPB PBx transposase fusion protein, or a ZNF-ssSPB transposase fusion protein.

19. A method of treating a disease or disorder in a subject in need thereof comprising administering to the subject at least one therapeutically effective dose of the cell of claim 10.

20. The method of any one of claims 17-19, wherein the disease or disorder is cancer, a liver disease or disorder, a urea cycle disorder, a metabolic liver disorder or a hemophilia disease.
